(19) United States
(12) Patent Application Publication
(10) Pub. No.: US 2016/0250245 A1
(43) Pub. Date: Sep. 1, 2016

(54) Methods and Pharmaceutical Composition for the Treatment of Polyomavirus Infections

(71) Applicants: INSERM (INSTITUT NATIONAL DE LA SANTÉ ET DE LA RECHERCHE MÉDICALE, Paris (FR); UNIVERSITÉ CLAUDE BERNARD - LYON 1, Villeurbanne (FR); ENS - ECOLE NORMALE SUPÉRIEURE DE LYON, Lyon (FR); CENTRE NATIONAL DE LA RECHERCHE SCIENTIFIQUE (CNRS), Paris (FR); HOSPICES CIVILS DE LYON, Lyon (FR)

(72) Inventors: Vincent Lotteau, Lyon Cedex 07 (FR); Patrice Andre, Lyon Cedex 07 (FR); Benoit De Chassey, Lyon Cedex 07 (FR); Laurene Meyniel-Schicklin, Lyon Cedex 07 (FR); Irene Pilar Castellanos, Lyon Cedex 07 (FR)

(21) Appl. No.: 15/030,416

(22) PCT Filed: Oct. 20, 2014
(86) PCT No.: PCT/EP2014/072398

§ 371 Date: Apr. 19, 2016

(30) Foreign Application Priority Data
Oct. 21, 2013 (EP) 13306443.6

(51) Int. Cl. A61K 31/7068; A61K 47/48

(52) U.S. Cl. CPC A61K 31/7068; A61K 47/48215; A61K 47/48038

(57) ABSTRACT

The present invention relates to methods and pharmaceutical compositions for the treatment of polyomavirus infections. In particular, the present invention relates to a method for treating a polyomavirus infection in a subject in need thereof comprising administering the subject with a therapeutically effective amount of gemcitabine.

### FIELD OF THE INVENTION

The present invention relates to methods and pharmaceutical composition for the treatment of polyomavirus infections

### BACKGROUND OF THE INVENTION

The process of organ transplantation has been revolutionized since the first successful kidney transplant more than five decades ago. Since then, the use of immunosuppressants, such as cyclosporine, have improved the outcome of transplants, but the process is still fraught with many challenges, such as the management of chronic and acute rejection, nephrotoxicity from immunosuppressant and antiviral drugs, and avoiding reactivated (or novel) infectious agents that could threaten the graft. For example, one of the problems that threatens kidney allograft survival is the development of polyomavirus-associated nephropathy (PVAN); also known as BKV associated nephropathy (BKVN)). Left untreated, PVAN can lead to a loss of the allograft. In kidney transplant recipients, current estimates of PVAN are about 1-10%, and graft losses range from 10-100% (depending on the drug regimen, monitoring, and interventions performed. The reduction or elimination of immunosuppressants in the treatment of BKVN is currently the most commonly used response to BKVN. Other treatments with antivirals and other compounds have proven to be less effective. Polyomavirus-associated pathologies such as PVAN or progressive multifocal leukoencephalopathy (PML) also cause significant morbidity or even mortality in other patients receiving immunosuppressive therapy (for example, for auto-immune disorders). Polyomaviruses are DNA-based (double-stranded DNA, −5000 base pairs, circular genome), small (40-50 nanometers in diameter), and icosahedral in shape, and do not have a lipoprotein envelope. Moreover, the genome possesses early and late genes, contributing to its complex transcription program. The main member of the family is polyomavirus BK or BK virus, which is widely distributed. Up to 80 percent of all tested subject are seropositive. This virus is believed to initiate infection in the urinary tract and then remain latent without disturbing its host, with occasional reactivation in the form of low-level shedding of virions in the urine (viruria). However, in immunocompromised individuals BKV (and the related JC polyomavirus) can cause significant morbidity or even mortality. In addition to kidney transplants, BKVN may occur in other immunosuppressed individual, for instance when undergoing haemopoietic stem cell transplants. BK virus infection may also be a factor in a fraction of haemorrhagic cystitis cases. Polyomaviruses are also potentially oncogenic (tumor-causing). So there is a need for better therapies to meet the treatment goals in polyomavirus infections, in particular BK virus infections.

### SUMMARY OF THE INVENTION

The present invention relates to methods and pharmaceutical compositions for the treatment of polyomavirus infections. In particular, the present invention is defined by the claims.

### DETAILED DESCRIPTION OF THE INVENTION

The present invention relates to methods and pharmaceutical compositions for the treatment of polyomavirus infections.

In particular, the present invention relates to a method for treating a polyomavirus infection in a subject in need thereof comprising administering the subject with a therapeutically effective amount of gemcitabine.

As used herein, the term “polyomavirus” has its general meaning in the art. Polyomavirus is the genus of the viruses within the family Polyomaviridae. Nine polyomaviruses have been discovered in humans: JCV, BKV, KI virus and WU virus, Merkel cell polyomavirus (MCV), Trichodysplasia sinulosa-associated polyomavirus (TSV), HPyV6, HPyV7, and HPyV9. Among these human polyomaviruses, JCV, BKV, and MCV cause serious complications and diseases.

In some embodiments, the method of the invention is particularly suitable for the treatment of BK virus infections.

As used herein, the term “BK virus” or ‘BKV” has its general meaning in the art and refers to the 4 BKV serotypes that are known (serotypes I-IV; e.g., Knowles et al., J. Med. Viol. 28: 118-123, 1989).

As used herein, an “active polyomavirus infection” refers to replication of a polyomavirus in a cell. “Polyomavirus replication” refers to any one or more of transcription of the late coding region of the polyomavirus genome, translation of the late coding region RNAs, virion production and virion release. “Reactivation of a polyomavirus” refers to the development of an active polyomavirus infection in a subject having a latent polyomavirus infection.

As used herein, a “latent polyomavirus infection” refers to polyomavirus infection that is not active. A subject having or suspected of having a latent polyomavirus infection includes a subject who has been exposed to a polyomavirus, and/or in whom the presence of polyomavirus DNA and/or anti-polyomavirus antibodies have been clinically detected.

The method of the invention may be carried out with any subject. The subject is preferably a mammal, more preferably a primate and more preferably still, a human. Subjects may be male or female and may be of any age, including prenatal (i.e., in utero), neonatal, infant, juvenile, adolescent, adult, and geriatric subjects. Thus, in some cases the subjects may be pregnant female subjects.

In some embodiments, the subject has or is suspected of having a latent polyomavirus infection.

In some embodiments, the subject has been diagnosed with an active polyomavirus infection

In some embodiments, the method is carried out with a subject at risk of developing a disease associated with a polyomavirus. Patients at risk of developing a polyomavirus-associated disease include individuals diagnosed with an active polyomavirus infection, individuals diagnosed with an active polyomavirus infection and are immunocompromised, and individuals that are immunocompromised and have or are suspected of having a latent polyomavirus infection. Immunocompromised individuals include but are not limited to AIDS patients; patients on chronic immunosuppressive treatment regimens, such as organ transplant patients; patients with cancer such as Hodgkin's disease or lymphoma; and patients with autoimmune conditions being treated with mycophenolate mofetil or a biologic such as natalizumab, rituximab, or efalizumab. Such autoimmune conditions include, but are not limited to multiple sclerosis (MS), rheumatoid arthritis (RA), and systemic lupus erythematosis (SLE). Elderly patients with weakened immune systems that have or are suspected of having a latent polyomavirus infection are also at risk of developing a disease associated with a polyomavirus.

In some embodiments, the subject has a cancer and is administered with a chemotherapeutic agent.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject is a transplant subject who is administered with an immunosuppressive agent. In some embodiments, the transplant subject has at least one transplanted organ selected from the group consisting of kidney, bone marrow, liver, lung, stomach, bone, testis, heart, pancreas and intestine.

In some embodiments, for the subject in need of an immunosuppressive agent, the compounds described herein may be administered in combination (concurrently or sequentially) with the immunosuppressive agent.

As used herein the term “immunosuppressive agent” refers to any agent that inhibits or prevents an activity of the immune system of the subject. Non-limiting examples of immunosuppressive agents include antibodies (e.g., fully human or humanized antibodies) that specifically bind to CD20, CD25 (e.g., basiliximab or daclizumab), or CD3 (e.g., muromonab); calcineurin inhibitors (e.g., ciclosporin, pimecrolimus, tacrolimus, sirolimus, and/or cyclosporine); interferons (e.g., interferon-β); steroids (e.g., any of the steroids known in the art or described herein); interleukin-1 receptor antagonists; myophenolate mofetil; Prograph®; azathioprine; methotrexate; and/or TNF-α binding proteins (e.g., antibodies and/or soluble TNF-α receptors, e.g., infliximab, etanercept, and/or adalimumab).

Treatment may be for any purpose, including the therapeutic treatment of previously infected subjects, as well as the prophylactic treatment of uninfected subjects (e.g., subjects identified as being at high risk for infection).

As used herein, the terms “treatment,” “treat,” and “treating” refer to reversing, alleviating, inhibiting the progress of a disease or disorder as described herein, or delaying, eliminating or reducing the incidence or onset of a disorder or disease as described herein, as compared to that which would occur in the absence of the measure taken. The terms “prophylaxis” or “prophylactic use” and “prophylactic treatment” as used herein, refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

The present invention also relates to a method for the prophylactic treatment of a disease associated with polyomaviruses in a subject in need thereof comprising administering the subject with a therapeutically effective amount of gemcitabine.

A number of diseases have been associated with polyomaviruses. In humans, progressive multifocal leukoencephalopathy (PML), neural tumors such as medulloblastoma, oligodendroglioma, astroglioma, and glioblastoma, and colorectal cancer have been associated with JC virus. PML has been detected in at-risk patients including AIDS patients; patients on chronic immunosuppressive treatment regimens, such as organ transplant patients; patients with cancer such as Hodgkin's disease or lymphoma; and patients with autoimmune conditions being treated with mycophenolate mofetil or a biologic such as natalizumab, rituximab, or efalizumab. BK virus has been associated with nephritis and/or nephropathy in patients who have undergone renal transplantation. BK virus has also been associated with prostate cancer. These polyomavirus-associated diseases are a significant part of graft loss. BK virus has also been associated with cystitis such as hemorrhagic and non hemorrhagic cystitis in patients who have undergone a bone marrow or stem cell transplant. MCV has been associated with Merkel cell carcinoma.

As used herein the term “gemcitabine” has its general meaning in the art and refers to 4-amino-1-[3,3-difluoro-4-hydroxy-5-(hydroxymethyl) tetrahydrofuran-2-yl]-1H-pyrimidin-2-one (CAS number 95058-81-4). Gemcitabine was earlier disclosed in U.S. Pat. No. 4,808,614. Gemcitabine is currently been marketed as a hydrochloride salt in a lyophilized formulation (Gemzar®) by Eli Lily and Company. Gemcitabine is a nucleoside analogue wherein the hydrogen atoms on the 2′ carbon of deoxycytidine are replaced by fluorine atoms. Typically, gemcitabine replaces one of the building blocks of nucleic acids, in this case cytidine, during DNA replication. The term also includes any gemcitabine derivative.

In some embodiments, the gemcitabine derivative refers to gemcitabine that has been derivatized with a lipophilic component that facilitates transport across the plasma (and/or other) membrane(s). Accordingly in some embodiments the gemcitabine derivative is typically a hydrophobic analog of gemcitabine, including lipophilic derivatives of gemcitabine.

In some embodiments, the gemcitabine derivative can be a compound of formula:

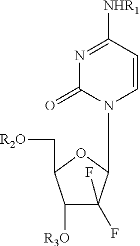

wherein R₁ and R₃ are hydrogen and R₂ is a C₁₈- or C₂₀-saturated and monounsaturated acyl group, or a pharmaceutically acceptable salt thereof.

In some embodiments, the gemcitabine derivative is gemcitabine-5′-elaidate, or gemcitabine-5′-elaidate ester (also referred to herein as gemcitabine-5′-elaidic acid, CP-4055, CP-4126, CO-1.01 and CO-101) which has the structure of formula:

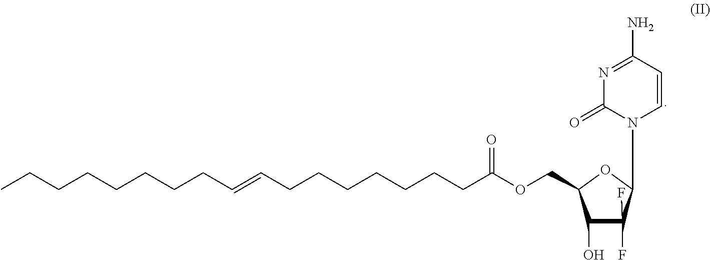

In some embodiments, the gemcitabine derivative is selected from the group consisting of the compounds having the general formula (I) to (VIII) as shown below, wherein R1 to R4 represent hydrogen or nitrogen protecting groups, especially esters, amides, acetals or ketals.

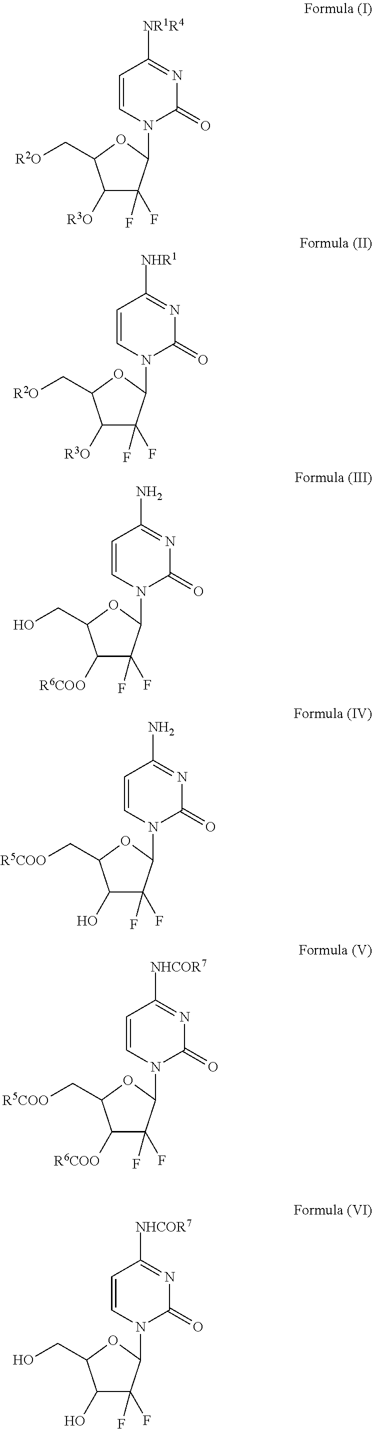

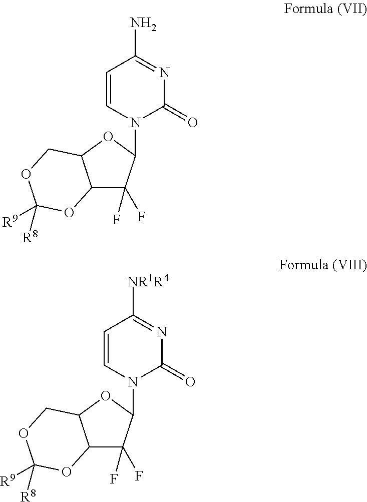

In some embodiments, R1, R2 and R3 are independently from each other selected from hydrogen and C1 to C30 saturated or monounsaturated or polyunsaturated acyl groups, preferably C8 to C26, and most preferably C12 to C24 saturated or monounsaturated or polyunsaturated acyl groups.

In some embodiments, gemcitabine derivatives include but are not limited to those described in WO/2010/039039 (ORAL FORMULATIONS OF GEMCITABINE DERIVATIVES).

In some embodiments, gemcitabine derivatives include also the phosphoramidite derivatives described in WO2012045999A1, and in particular, the derivative selected from the group consisting of 2′-Deoxy-2̂2′-difiuoro-D-cytidme-5,0-bis(ethoxy-L-alaninyl)-phosphate; 2′-Deoxy-2̂2′-difluoro-D-cytidme-5′-0-bis(benzoxy-L-alaiiinyl)-phosphate; 2′-Deoxy-2 2′-difluoro-D-cytidme-5′-0-bis(cyclohexoxy-L-alaninyl)-phosphate; 2′-Deoxy-2̂2̂-difluoro-D-cytidme-5′-0-bis(2,2-dimethylpropoxy-L-alani phosphate; and 2′-Deoxy-2 2′-difluoro-D-cvtidme-5′-0-bis(iso-propoxy-L-alaninyl)-phosphate.

In some embodiments, gemcitabine derivatives include those described in WO 1998032762 and in WO 2013044811.

In some embodiments, gemcitabine derivatives include pegylated gemcitabine derivatives such as described in WO2012098557.

Typically gemcitabine is administered to the subject with a therapeutically effective amount. By a “therapeutically effective amount” of gemcitabine as above described is meant a sufficient amount of gemcitabine to treat a polyomavirus infection at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination with the specific agonist employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, gemcitabine is administered to the subject in manner to reach an active concentration in the namolar range. In some embodiments, the gemcitabine is administered to the subject to reach a concentration of about 1; 1.05; 1.1; 1.15; 1.2; 1.25; 1.3; 1.35; 1.4; 1.45; 1.5; 1.55; 1.6; 1.65; 1.7; 1.75; 1.8; 1.85; 1.9; 1.95; 2; 2.05; 2.1; 2.15; 2.2; 2.25; 2.3; 2.35; 2.4; 2.45; 2.5; 2.55; 2.6; 2.65; 2.7; 2.75; 2.8; 2.85; 2.9; 2.95; 3; 3.05; 3.1; 3.15; 3.2; 3.25; 3.3; 3.35; 3.4; 3.45; 3.5; 3.55; 3.6; 3.65; 3.7; 3.75; 3.8; 3.85; 3.9; 3.95; 4; 4.05; 4.1; 4.1; 4.15; 4.2; 4.25; 4.3; 4.35; 4.4; 4.45; 4.5; 4.55; 4.6; 4.65; 4.7; 4.75; 4.8; 4.85; 4.9; 4.95; 5; 5.05; 5.1; 5.15; 5.2; 5.25; 5.3; 5.35; 5.4; 5.45; 5.5; 5.55; 5.6; 5.65; 5.7; 5.75; 5.8; 5.85; 5.9; 5.95; 6; 6.05; 6.1; 6.15; 6.2; 6.25; 6.3; 6.35; 6.4; 6.45; 6.5; 6.55; 6.6; 6.65; 6.7; 6.75; 6.8; 6.85; 6.9; 6.95; 7; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10 nM.

Gemcitabine may be combined with pharmaceutically acceptable excipients, and optionally sustained-release matrices, such as biodegradable polymers, to form pharmaceutical compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local, intravesical or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and vesical and rectal administration forms.

In particular, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Gemcitabine can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various proportions of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

Gemcitabine may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Effect of gemcitabine on BKV (Dunlop) replication in Vero cell line. Panel A shows a typical experiment done in quadruplicate. The upper dashed lines indicate the mean+/−one standard deviation of the viral concentration in the supernatant of infected and non-treated wells. The lower dashed lines indicate the mean+/−one standard deviation of the remaining viral genome in the supernatant as measured in the last wash of the cells after infection. The line shows the effect of increasing concentrations of gemcitabine on virus production in cell supernatants. Vertical bars indicate the SE. Panel B shows the LDH activity in arbitrary units in culture supernatants (open square) and in cell lysates (closed square) determined after two days of treatment in the same wells as in A. Vertical bars indicate the standard deviations. LDH activity measured simultaneously on infected and uninfected cells showed no impact on cell lysis or viability (data not shown).

FIG. 2: Comparison of the effect of gemcitabine and cidofovir on the BKV (Dunlop strain) replication in RPTEC primary cells. Cells were cultured, infected and treated with gemcitabine (A) or cidofovir (B) at the indicated final concentrations as described in the material and methods section. Supernatants were collected at days 3 (black histogram) and 5 (grey histogram) post-infection and BKV load measured by qPCR. Results of typical experiments done in quadruplicate+ or − standard error are shown.

FIG. 3: Analysis of RPTEC primary cells viability after 5 days exposure to gemcitabine. Panel A shows the analysis of infected cell viability and growth, in presence of the indicated gemcitabine concentrations, measured by LDH activity in the supernatant, close triangles, and in cell, close squares, harvested at day 5 post-infection. Panel B shows the cell viability as assessed by the cellular LDH activity (black histogram) and the intracellular DNA synthesis as measured by BrdU incorporation (grey histogram). RPTEC cells were mock infected or infected as described and infected cells were treated with the indicated concentrations of gemcitabine and harvested for assays at day 5 post-infection. Representative experiments done in quadriplicate are shown. Vertical bars indicate standard errors.

FIG. 4: Effects of gemcitabine treatment on the expression of T antigen and VP-1 in RPTEC cells. Cells were cultured, infected and treated with gemcitabine at the indicated concentrations as described in the material and methods section. After 5 days of treatment cells were fixed and stained with fluorescent antibodies against Ag T (left panel) or against VP-1 (right panel) and analyzed as described. Nuclei were stained with Hoescht reagent (lower panel).

### EXAMPLE

### Material & Methods

Cells, Virus and Reagents

Primary human renal proximal tubule epithelial cells (RPTEC) (Lonza, www.lonza.com) were propagated as described by the manufacturer with and without grown factors. Cercopithecus aethiops renal cells (Vero cell line) were cultured in the base medium 199 (Lonza, CH), supplemented with inactivated fetal bovine serum (FBS Brazilian origin, Lonza) and penicillin-streptomycin 10000 U/μg to a final concentration of 6% and 2% respectively. Stock of Vero cells was stored in liquid nitrogen in aliquots of 2.75×10⁶ cells. Stock BKV, Dunlop strain, was kept frozen in aliquots at 1.19×10¹⁰ genomes/mL. Gemcitabine hydrochloride and cidofovir hydrate were purchased from Sigma Aldrich (Saint-Quentin Fallavier, France) and dissolved in PBS and deionized water respectively, at 10 mM.

Infection of Vero Cell Line and Gemcitabine-Treatment

For each assay, one cell aliquot was thawed and cells cultured for three days in one 25 cm² culture flask. Cells were then trypsinized, washed, resuspended and seeded at 1×10⁴ cells/well in 1004 medium in flat bottom 96-well microplates. After 24 h incubation, cells at 75% confluence were infected with 1×10⁹ genomes per well in BK medium (199 medium with 2% FBS, 2% HEPES, 1% penicillin-streptomycin 10000 U/μg final concentration). After 4 h incubation (37° C., 5% CO₂), medium was removed and replaced with 200 μL of fresh BK medium after four washes. 72 h later, medium was replaced with fresh BK medium with or without gemcitabine at the indicated final concentrations. After 48 h incubation, cell culture supernatants were harvested and frozen at −80° C.

Infection of Primary Human Renal Proximal Tubule Epithelial Cell (RPTEC)

For each assay, one cell aliquot containing 2×10⁶ cells was thawed and cells were cultured for six days in one 75 cm² culture flask. Cells were then trypsinized, washed, resuspended and seeded at 5×10⁴ cells/well in 1 ml medium in flat bottom 24-well collagen-coated microplates. After 48 h incubation, medium was replaced with fresh REBM base medium without grown factors and supplemented with inactivated fetal bovine serum (FBS Brazilian origin, Lonza) and penicillin-streptomycin 10000 U/μg to a final concentration of 2% and 1% respectively. 48 h later, cells at 95% confluence were infected with 1×10⁹ genomes per well. After 4 h incubation (37° C., 5% CO₂), medium was removed and replaced with 1 ml of fresh medium without grown factors after two washes with and without gemcitabine or cidofovir at the indicated concentrations. 72 h later, at day 3 of treatment, supernatants were harvested and frozen at −80° C. and medium was replaced with fresh medium without grown factors and with the indicated concentrations of gemcitabine or cidofovir. After 48 h incubation, at day 5 of treatment, cell culture supernatants were harvested and frozen at −80° C.

Quantification of BKV DNA

Extracellular DNA was extracted from 100 μL frozen supernatants using the automated nucleic acids extractor Nuclisens Easymag (BioMérieux, Marcy l'Etoile, France) following the manufacturer's Generic 2.0.1 procedure. Extracts were resuspended in a final volume of 25 μL buffer 3.

To quantify extracellular BKV DNA load, a quantitative PCR (qPCR) was done using the following primers (Eurofins MWG Operon) and probe (Applied Biosystem, UK):

BK_1:
(SEQ ID NO: 1)
5′ AGC-AGG-CAA-GGG-TTC-TAT-TAC-TAA-AT 3′
BK_2:
(SEQ ID NO: 2)
5′ GAA-GCA-ACA-GCA-GAT-TCT-CAA-CA 3′
Probe BK:
(SEQ ID NO: 3)
FAM-5′ CCT-AAA-GAC-TTT-CCC-TCT-GAT 3′

Reactions were performed in a total volume of 25 μl with 5 μL of DNA extract, 10 μL TaqMan Universal PCR Master Mix (Applied Biosystems, Hoffmann La Roche, Meylan, France), 6.5 μL H₂O, 2.5 μL of each primer at 30 μM and 1 μL of probe at 10 μM.

Amplification was performed using the Rotor-Gene 6000 (Corbett Life Science) with the following PCR program: 50° C., 2 min; denaturation at 95° C., 10 min; and 45 cycles of 95° C., 15 sec; 60° C., 60 sec. Quantification was calculated with an external calibration curve using BKV plasmid constructs.

Immunofluorescence Staining, Microscopy and Digital Image Processing

RPTEC cells were washed with phosphate-buffered saline (PBS), fixed in 100% methanol for 15 min at −20° C., and blocked with PBS containing 2% bovine serum albumin (BSA) for 30 min at room temperature, followed by primary and secondary antibody incubation for 1 h at 37° C. Primary antibodies were monoclonal anti-simian virus 40 (SV40) T antigen (Ab-2) Mouse mAb (PAb416) (1:50, www.merckmillipore.com) and monoclonal mouse Anti-BKV VP1 mAb (1:1000, Abnova, www.abnova.com). The secondary antibody was Alexa Fluor 488 Goat Anti-Mouse IgG (H+L) Antibody (1:1000, www.lifetechnologies.com). Nuclei were labeled with Hoechst 33342 (1:1000, www.piercenet.com). Images were collected using a microscope Leica DM/IRB equipped with the camera Axiocam Cm1.

Cytotoxicity Detection by LDH and BrdU Assays

Cytotoxicity was analyzed by two colorimetric assays using the “Cytotoxicity Detection Kit (LDH)” and “Cell proliferation ELISA, BrdU” (Roche Diagnostics, Meylan, France).

The quantity of LDH was measured in cell culture supernatants and cells from every wells used for the quantification of virus replication including infected and non-infected wells and in presence or not of gemcitabine at the end of the test period according to the manufacturer's protocol. Absorbance at 492 nm (sample) and at 620 nm (background) was recorded 30 min after addition of the reaction mixture. Medium alone served as blank. Measure of LDH activity in culture supernatants was used to monitoring cell lysis and the activity in cell lysates to monitoring cell viability and mass at 72 and 120 h p.i

DNA synthesis was quantified by the colorimetric measurement of BrdU incorporation into DNA in proliferating cells. BrdU incorporation was measured at 72 et 120 h p.i, according to the manufacturer protocol. Absorbance was determined at 450 nm (sample) and 620 nm (background) 10 min after addition of the substrate. Lectures were done using a 96 well-plates ELISA reader (SunRise, Tecan) and were processed by Magellan data analyse software.

Results:

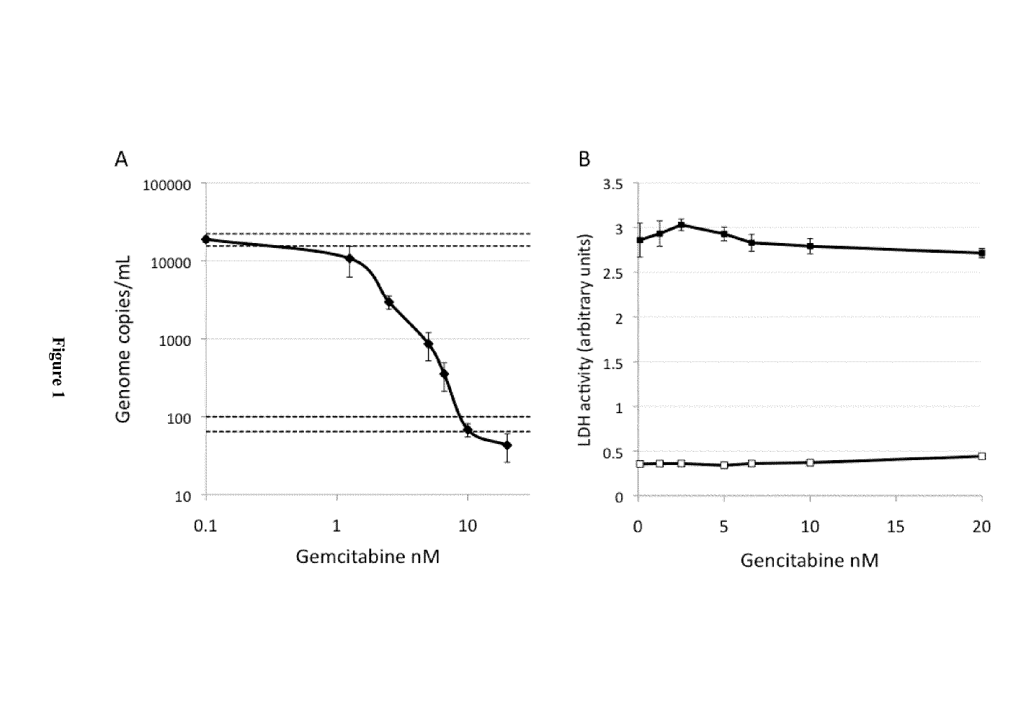

In Vero cells, gemcitabine inhibits viral replication in the nanomolar range (FIG. 1). Indeed concentration as low as 10 nM totally inhibited viral replication in two days. The IC50 ranges from 1.5 to 2 nM and the IC90 from 2.5 to 5 nM. At these concentrations and up to 20 nM of gemcitabine no significant modification of cell lysis or viability was observed in the conditions of the assay. The IC50 specificity index is thus ≧10 in this cell culture system.

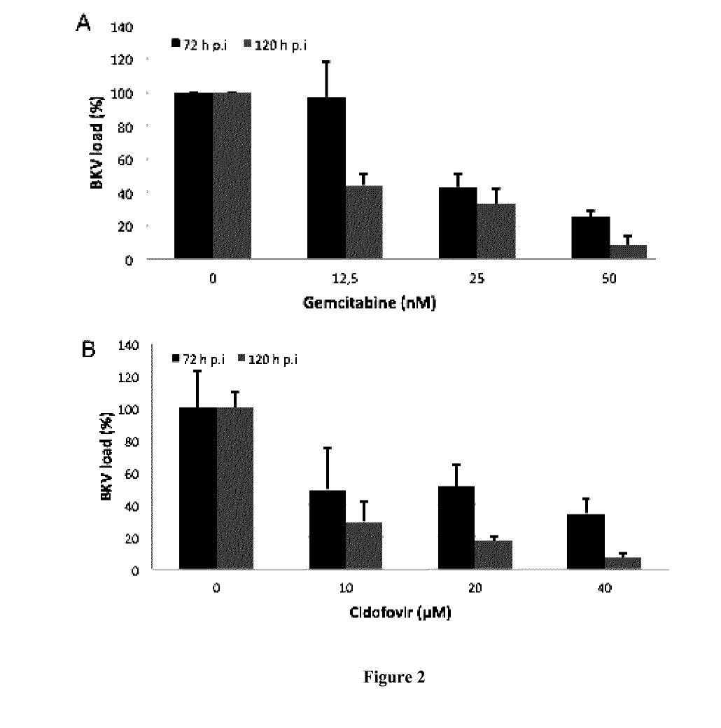

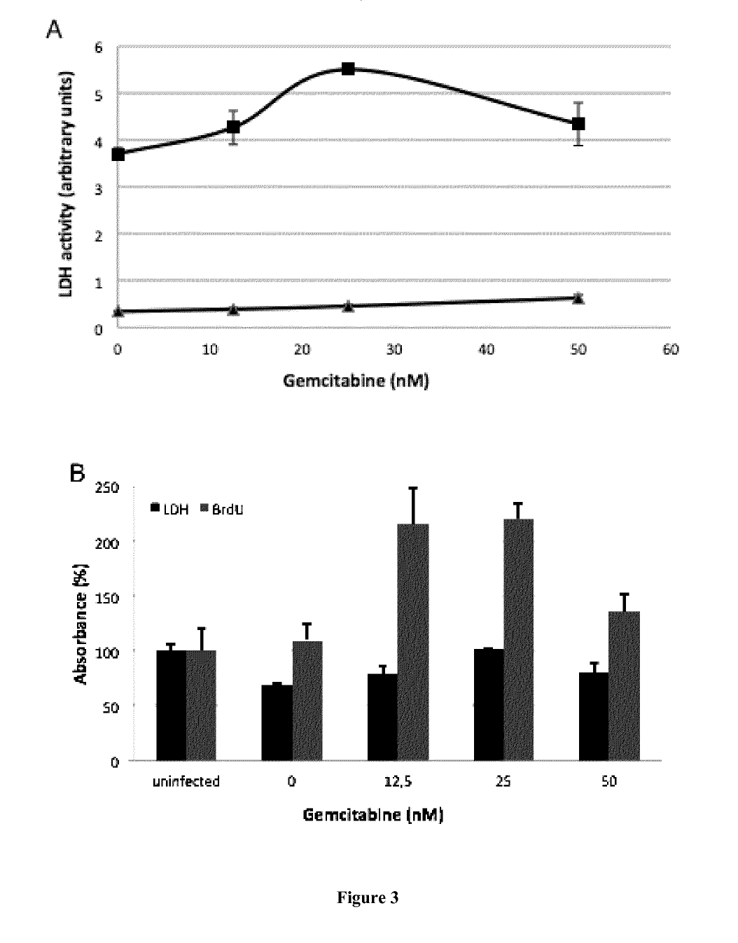

In RPTEC, gemcitabine also inhibits viral replication. The inhibitory effect of gemcitabine was higher after 5 days than after 3 days of treatment (FIG. 2A). IC50 and IC90 range in the 10 nM and 50 nM respectively. Interestingly, when tested in the same conditions, the reference antiviral cidofovir repressed BKV replication in the μM range (FIG. 2B). At these concentrations, no toxicity was observed; on the contrary treatment with gemcitabine at effective antiviral concentrations (up to 10 nM) could suppress the inhibitory effect of viral infection on the cellular growth measured by the intracellular LDH activity (FIG. 3). Similar effect was observed when measuring total cellular DNA synthesis by BrdU incorporation (FIG. 3B). However, higher concentrations of gemcitabine in the 50 nM range seemed to slow down both cellular growth and DNA synthesis.

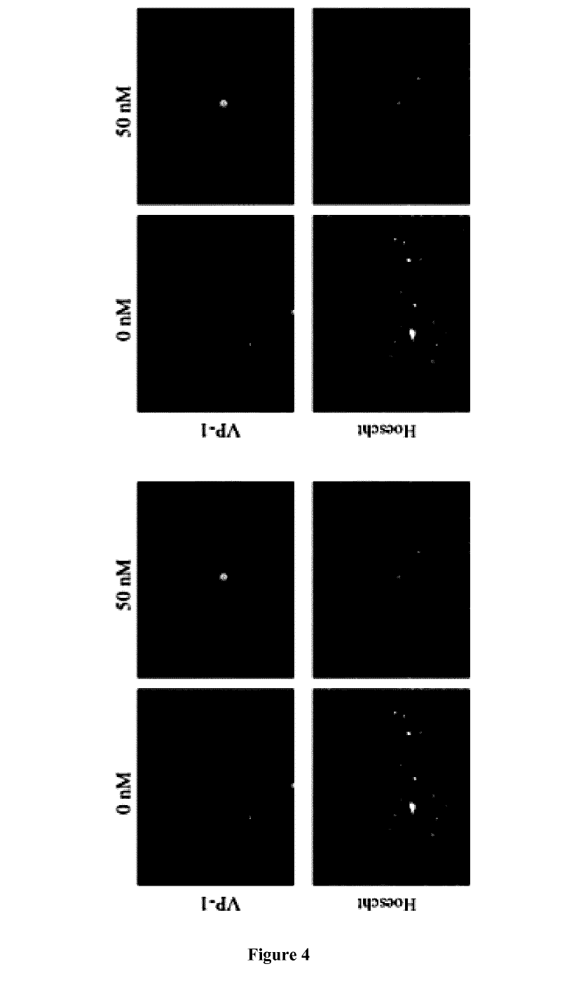

The antiviral effect of gemcitabine on the viral protein synthesis was also analyzed by immunofluorescence. The expression of the early protein antigen T and of the late capsid protein VP-1 was inhibited as shown in FIG. 4.

In summary, gemcitabine potently represses BKV replication in two different cellular models, including primary renal epithelial cells, at concentrations in the 10 nM range, which are concentrations 100 to 1000 times lower than concentrations needed in tumor treatment (Yilmaz et al. 2004; Analytical Biochemistry, 332:234-237).

### REFERENCES

Throughout this application, various references describe the state of the art to which this invention pertains. The disclosures of these references are hereby incorporated by reference into the present disclosure.

1. A method of treating a polyomavirus infection in a subject in need thereof comprising administering to the subject a therapeutically effective amount of gemcitabine or a gemcitabine derivative.

2. The method of claim 1 wherein the polyomavirus is selected from the group consisting of JCV, BKV, KI virus, WU virus, Merkel cell polyomavirus (MCV), Trichodysplasia sinulosa-associated polyomavirus (TSV), HPyV6, HPyV7, and HPyV9.

3. The method of claim 1 wherein the polyomavirus is BK virus.

4. The method of claim 1 wherein the subject has or is suspected of having a latent polyomavirus infection.

5. The method of claim 1 wherein the subject has been diagnosed with an active polyomavirus infection

6. The method of claim 1 wherein the subject is at risk of developing a disease associated with a polyomavirus, and is selected from the group consisting of individuals diagnosed with an active polyomavirus infection, individuals who are immunocompromised and diagnosed with an active polyomavirus infection, and individuals who are immunocompromised and have or are suspected of having a latent polyomavirus infection.

7. The method of claim 6 wherein the individuals who are immunocompromised are selected from the group consisting of AIDS patients; patients on chronic immunosuppressive treatment regimens, patients with cancer, patients with autoimmune conditions being treated with mycophenolate mofetil or a biologic, and elderly patients with weakened immune systems that have or are suspected of having a latent polyomavirus infection.

8. The method of claim 7 wherein the patients on chronic immunosuppressive treatment regimens are organ transplant patients to whom an immunosuppressive agent is administered.

9. The method of claim 8 wherein the organ transplant patients have at least one transplanted organ selected from the group consisting of kidney, bone marrow, liver, lung, stomach, bone, testis, heart, pancreas and intestine.

10. The method of claim 6 wherein the gemcitabine or gemcitabine derivative is administered in concurrent or sequential combination with an immunosuppressive agent.

11. The method of claim 10 wherein the immunosuppressive agent is selected from the group consisting of antibodies that specifically bind to CD20, CD25 or CD3; calcineurin inhibitors interferons steroids; interleukin-1 receptor antagonists; myophenolate mofetil; Prograph®; azathioprine; methotrexate; and TNF-α binding proteins.

12. A method for the prophylactic treatment of a disease associated with polyomaviruses in a subject in need thereof comprising administering to the subject a therapeutically effective amount of gemcitabine.

13. The method of claim 12 wherein the disease associated with polyomaviruses is selected from the group consisting of progressive multifocal leukoencephalopathy (PML), neural tumors, colorectal cancer, prostate cancer, Merkel cell carcinoma, nephritis and/or nephropathy in patients who have undergone renal transplantation, hemorrhagic-cystitis in patients who have undergone a bone marrow or stem cell transplant, and non hemorrhagic cystitis in patients who have undergone a bone marrow or stem cell transplant.

14. The method according to claim 1, wherein the gemcitabine derivative is a compound of formula: 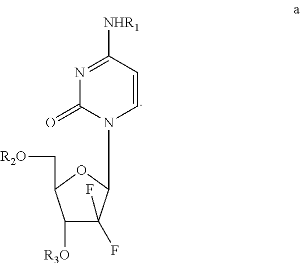
wherein R1 and R3 are hydrogen and R2 is a C18- or C20-saturated or monounsaturated acyl group, or a pharmaceutically acceptable salt thereof.

15. The method according to claim 1, wherein the gemcitabine derivative is gemcitabine-5-elaidate, or gemcitabine-5-elaidate ester which has the structure of formula:

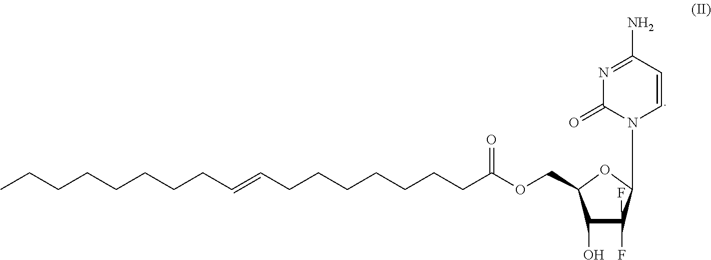

16. The method according to claim 1, wherein the gemcitabine derivative is selected from the group consisting of compounds having the general formula (I) to (VIII): 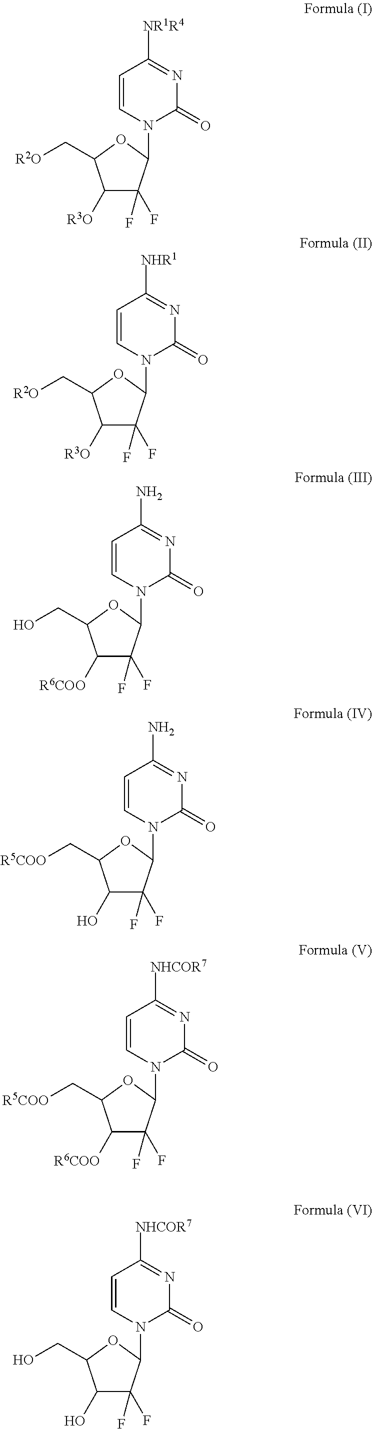 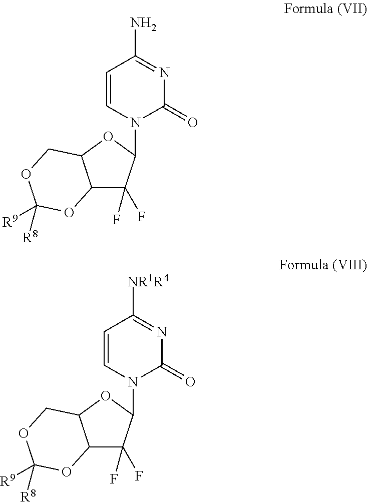
wherein R1 to R4 represent hydrogen or nitrogen protecting groups.

17. The method of claim 16 wherein R1, R2 and R3 are independently selected from hydrogen and C1 to C30 saturated, monounsaturated or polyunsaturated acyl groups.

18. The method according to claim 1, wherein the gemcitabine derivative is selected from the group consisting of 2′-Deoxy-2̂2′-difiuoro-D-cytidme-5,0-bis(ethoxy-L-alaninyl)-phosphate; 2′-Deoxy-2̂2′-difluoro-D-cytidme-5′-0-bis(benzoxy-L-alaiiinyl)-phosphate; 2′-Deoxy-2,2′-difluoro-D-cytidme-5′-0-bis(cyclohexoxy-L-alaninyl)-phosphate; 2′-Deoxy-2̂2̂-difluoro-D-cytidme-5′-0-bis(2,2-dimethylpropoxy-L-alani phosphate; and 2′-Deoxy-2 2′-difluoro-D-cvtidme-5′-0-bis(iso-propoxy-L-alaninyl)-phosphate.

19. The method according to claim 1, wherein the gemcitabine derivative is a pegylated gemcitabine derivative.

20. The method according to claim 1, wherein the gemcitabine or gemcitabine derivative is administered to the subject in manner to reach an active concentration in the nanomolar range.

21. The method of claim 20 wherein the gemcitabine or gemcitabine derivative is administered to the subject to reach a concentration of about 1; 1.05; 1.1; 1.15; 1.2; 1.25; 1.3; 1.35; 1.4; 1.45; 1.5; 1.55; 1.6; 1.65; 1.7; 1.75; 1.8; 1.85; 1.9; 1.95; 2; 2.05; 2.1; 2.15; 2.2; 2.25; 2.3; 2.35; 2.4; 2.45; 2.5; 2.55; 2.6; 2.65; 2.7; 2.75; 2.8; 2.85; 2.9; 2.95; 3; 3.05; 3.1; 3.15; 3.2; 3.25; 3.3; 3.35; 3.4; 3.45; 3.5; 3.55; 3.6; 3.65; 3.7; 3.75; 3.8; 3.85; 3.9; 3.95; 4; 4.05; 4.1; 4.1; 4.15; 4.2; 4.25; 4.3; 4.35; 4.4; 4.45; 4.5; 4.55; 4.6; 4.65; 4.7; 4.75; 4.8; 4.85; 4.9; 4.95; 5; 5.05; 5.1; 5.15; 5.2; 5.25; 5.3; 5.35; 5.4; 5.45; 5.5; 5.55; 5.6; 5.65; 5.7; 5.75; 5.8; 5.85; 5.9; 5.95; 6; 6.05; 6.1; 6.15; 6.2; 6.25; 6.3; 6.35; 6.4; 6.45; 6.5; 6.55; 6.6; 6.65; 6.7; 6.75; 6.8; 6.85; 6.9; 6.95; 7; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10 nM.

22. The method of claim 7, wherein the patients with cancer are Hodgkin's disease patients or lymphoma patients.

23. The method of claim 7, wherein the biologic is natalizumab, rituximab, or efalizumab,

24. The method of claim 11, wherein the calcineurin inhibitor is selected from the group consisting of ciclosporin, pimecrolimus, tacrolimus, sirolimus and cyclosporine.

25. The method of claim 13, wherein the neural tumor is a medulloblastoma, an oligodendroglioma, an astroglioma or a glioblastoma.

26. The method of claim 16, wherein the nitrogen protecting group is an ester, an amide, an acetal or a ketal.

27. The method of claim 17, wherein the C1 to C30 saturated monounsaturated or polyunsaturated acyl groups are
i) C8 to C26 saturated, monounsaturated or polyunsaturated acyl groups or
ii) C12 to C24 saturated, monounsaturated or polyunsaturated acyl groups.


SEQUENCE LISTING (3 sequences)

SEQ ID NO: 1 - DNA, 26 bp, Artificial
  Synthetic oligonucleotide primer
agcaggcaagggttctattactaaat

SEQ ID NO: 2 - DNA, 23 bp, Artificial
  Synthetic oligonucleotideprimer
gaagcaacagcagattctcaaca

SEQ ID NO: 3 - DNA, 21 bp, Artificial
  Synthetic oligonucleotideprobe
cctaaagactttccctctgat